(19) United States
(12) Patent Application Publication
(10) Pub. No.: US 2009/0318366 A1
(43) Pub. Date: Dec. 24, 2009

(54) CHOLESTEROL LOWERING PROTEIN HYDROLYSATES

(75) Inventors: Luppo Edens, Rotterdam (NL); Daniel Raederstorff, Flaxlanden (FR)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/293,942

(22) PCT Filed: Mar. 21, 2007
(86) PCT No.: PCT/EP2007/052694

§ 371 Date: Feb. 23, 2009

(30) Foreign Application Priority Data
Mar. 22, 2006 (EP) 06111561.4
Oct. 19, 2006 (EP) 06122613.0

(51) Int. Cl. A61K 38/08; C07K 5/00; C07K 7/00; C12P 21/02; A61K 38/07

(52) U.S. Cl. 514/16; 530/330; 530/329; 530/328; 530/327; 435/68.1; 514/18; 514/17

(57) ABSTRACT

The present invention relates to the production of novel peptides which have a length of between 4 and 8 amino acids and comprises the amino acid sequence -Xaa-Xbb-Xcc-Xdd- or -Xbb-Xcc-Xdd-Xaa-, whereby Xaa and Xcc can be His (H), Arg (R) or Lys (K), and Xbb and Xdd can be Pro (P) or Gly (G).

### FIELD OF THE INVENTION

The present invention relates to the production of novel peptides.

### BACKGROUND OF THE INVENTION

In the Western world, more than 95% of the dietary fat is long-chain triglycerides, with the remaining part largely made up of phospholipids, fatty acids and cholesterol. Because elevated plasma cholesterol levels (hypercholesterolemia or hyperlipidemia) are widely regarded as a primary risk factor for artherosclerosis and coronary heart disease, lowering of cholesterol intake is of broad medical importance. Plasma cholesterol levels are determined by the balance between diet, cholesterol biosynthesis, cholesterol secretion in bile acids from the liver, and cholesterol absorption and reuptake by the small intestine.

The overall whole-body cholesterol balance is kept mainly by matching fecal losses with endogenous cholesterol synthesis. Because of its very low water solubility, cholesterol uptake is completely dependent upon special transport mechanisms. The successive steps in the process of intestinal cholesterol digestion and absorption have been reviewed by Ros (Artherosclerosis 2000, 151, 357-379). Briefly, the peristaltic action of the stomach leads to an initial emulsion of water-soluble and non-water soluble dietary compounds. This emulsion is stabilized by a.o. dietary phospholipids and free fatty acids and monoglycerides formed by the gastric lipase. Once in the duodenum, the stomach contents interacts with specific receptors to stimulate the secretion of bile and pancreatic juice. Major components of bile are bile acids, phospholipids, cholesterol, proteins and bile pigments (Tuchweber et al., Nutr. Res. 1996, 16, 1041-1080). The pancreatic juice supplies the lipase that leads to a further breakdown of dietary fats as well as proteases leading to a breakdown of the dietary proteins into peptides and free amino acids. Stimulated by an increasing pH value due to bicarbonate secretion and the increasing concentrations of bile, lipophilic products present in the stomach content become increasingly solubilized in the form of micelles. The formation of these bile acid micelles are the first steps in a complex process that eventually results in the transfer of cholesterol and the hydrolysed dietary fats over the intestinal wall to end up in the blood.

A relatively large amount of cholesterol in the body is of dietary origin and taken up from the gut in the form of bile salt micelles or small unilamellar phospholipid vesicles according to the above described mechanism. By interfering the process of micellar cholesterol solubilization, the uptake of dietary cholesterol can be limited and hereby plasma cholesterol levels. WO 01/24789 describes socalled beta-peptides that are suitable as active substances that reduce cholesterol- and lipid-uptake from the gut. Beta-peptides consist of beta-amino acids which have an amino group in the beta-position. Beta-peptides form a more stable helix than natural (alpha) peptides do, but, more importantly, beta-peptides are stable against proteolytic degradation. According to WO 01/24789 the beta-peptides should be at least about 6 residues long and have at least two positive charges or should be amphipatic. The mechanism provided is that these beta-peptides act as a precipitant for cholesterol and lipids comprising micelles and vesicles having a negative surface charge. Their data show that polylysine as well as beta-peptides lead to an effective cholesterol precipitation whereas free lysine and free arginine, carrying only a single positive charge.

Apart from such beta-peptides, also intact dietary proteins such as soybean protein and their hydrolysates are known to influence serum cholesterol levels. Cholesterol lowering effects have been described for soybean, casein and whey hydrolysates. From the whey protein beta-lactoglobulin specific hypocholesterolemic peptides have been described (Nagaoka et al., Biochem. Biophys. Res. Commun. 2001, 281, 11-17). Among the four peptides able to inhibit cholesterol absorption in vitro, peptide Ile-Ile-Ala-Glu-Lys could be shown also to be active in vivo. The latter peptide was selected for the in vivo test because only this peptide has no peptic or tryptic digestive site. Another publication (Yoshikawa et al., BioFactors 2000, 12, 143-146) mentions alpha-lactotensin, the beta-lactoglobulin derived peptide His-Ile-Arg-Leu, as a peptide capable of reducing serum cholesterol. The same publication refers to Leu-Pro-Tyr-Pro-Arg as a serum cholesterol reducing peptide obtained from soybean glycinin. In another study (Dae Young Kwon et al., Food Sci Biotechnol 2002, 11 (1) 55-61)) the hypocholesterolemic quality of the latter peptide was confirmed. According to these and other data, hydrophobicity is a factor of prime importance for bile binding and thus cholesterol lowering activity of these cholesterol loweing peptides. The presence of the C-terminal Arg residue was considered not so critical for the peptide's hypocholesterolemic activity.

Also green tea catechins and calcium from dairy products have been mentioned as cholesterol uptake inhibitors. Epigallocatechin gallate (EGCG) is the major catechin found in green tea. In humans, tea catechins have been shown to reduce body weight, visceral fat mass, and plasma cholesterol, insulin, and glucose levels. Additionally, several animal studies demonstrated that catechins inhibited cholesterol absorption and lowered plasma cholesterol levels. The current explanation is that epicatechins increase the fecal excretion of cholesterol and total lipids. In a very recent human, randomized crossover study (Am J Clin Nutr 2007; 85:678-687), it became clear that increased calcium intake from dairy products attenuates postprandial lipidemia, probably because of reduced fat absorbtion. Presumably the calcium acts by forming insoluble fatty acid soaps or by binding to the bile acids, hereby impairing micel formation. Calcium supplements in the form of calcium carbonate were found to be non effective suggesting that another anion as, for example, phosphate is required.

### SUMMARY OF THE INVENTION

The present invention relates to the novel peptides which have a length of between 4 and 8 amino acids and comprises the amino acid sequence -Xaa-Xbb-Xcc-Xdd- or -Xbb-Xcc-Xdd-Xaa-, whereby Xaa and Xcc can be His (H), Arg (R) or Lys (K), and Xbb and Xdd can be Pro (P) or Gly (G). Preferably the novel peptides have a length of between 4 and 6 amino acids, more preferably 4 or 5 amino acids. Preferably this peptide has a positive charge. In a preferred embodiment Xbb or Xdd is Pro (P), preferably Xbb and Xdd are Pro (P). In another preferred embodiment Xaa and/or Xcc is Arg (R). Preferred peptides are R-P-R-P-R-P-S-F-I-A-I-P-P-K, R-P-R-P-R-P-S-F, R-P-R-P-R-P-S, R-P-R-P-S, R-P-R-P, K-P-R-P, R-G-R-P-S, H-P-H-P-H, H-P-H-P-H-L-S-F, K-P-K-P-S, K-G-K-P, K-P-K-P, R-P-R-P, R-G-R-G P-R-P-K, P-K-G-K, P-R-G-R-, G-R-G-R, G-K-G-K, P-K-P-K and P-R-P-R.

The present invention also relates to a protein hydrolysate comprising the novel peptides which have a length of between 4 and 8 amino acids and comprising the amino acid sequence -Xaa-Xbb-Xcc-Xdd- or -Xbb-Xcc-Xdd-Xaa-. Preferably the protein hydrolysate has a DH of 5 to 50%, more preferably 10 to 40% and most preferably a DH of 20 to 35%. Preferably the protein hydrolysate or peptide mixture has an enhanced proline content. A preferred protein hydrolysate or peptide mixture has a molar proline content (on basis of total amino acid residues present) of more than 10%, more preferred protein hydrolysate or peptide mixture has a molar proline content of more than 20%, even more preferred protein hydrolysate or peptide mixture has a molar proline content of more than 30%. Hydroxyproline residues, as present in collagen and gelatin, are excluded from these proline contents. Furthermore the present invention relates to a peptide mixture comprising the novel peptides which have a length of between 4 and 8 amino acids and comprising the amino acid sequence -Xaa-Xbb-Xcc-Xdd- or -Xbb-Xcc-Xdd-Xaa-. Preferably the peptide mixture or hydrolysate of the invention comprises at least 1 mg/g protein of the peptide of the invention, more preferably at least 4 mg/g, even more preferably at least 10 mg/g protein, still more preferably at least 25 mg/g protein and most preferably at least 50 mg/g protein. In general the peptide mixture or hydrolysate will have less than 300 mg/g protein of the peptide of the invention. In case of synthetically produced peptide or purified isolated peptide an amount of 1 g/g protein is possible.

This peptide mixture or hydrolysate of the invention preferably comprises at least 30 wt % (dry matter) peptides having a MW of less than 2000 Da, more preferably 35 to 90% wt (dry matter) of the peptide mixture are peptides having a MW of less than 1500 Da.

The present invention also relates to a enzymatic process to produce the novel peptides which have a length of between 4 and 8 amino acids and comprising the amino acid sequence -Xaa-Xbb-Xcc-Xdd- or -Xbb-Xcc-Xdd-Xaa-, preferably by enzymatic hydrolysis of a protein source.

Furthermore the present invention relates to the novel peptides that are produced using chemical, chemo-enzymatical or peptide fermentation techniques.

The present invention further comprises a method of treatment or preventing of hypercholesterolemia, artherosclerosis, coronary heart disease or increasing plasma HDL (High Density Lipoprotein) which comprises the intake of a peptide which have a length of between 4 and 8 amino acids and comprising the amino acid sequence -Xaa-Xbb-Xcc-Xdd- or -Xbb-Xcc-Xdd-Xaa-Xaa-, a protein hydrolysate which comprises this peptide, or a peptide mixture comprising this peptide or the intake of a nutraceutical, food or feed product which comprises said peptide, said protein hydrolysate or said peptide mixture.

Furthermore the present invention provides a method for the preparation of a nutraceutical, dietary supplement, food or feed product for the treatment or preventing of hypercholesterolemia, artherosclerosis, coronary heart disease or increasing plasma HDL (High Density Lipoprotein) whereby a peptide which has a length of between 4 and 8 amino acids and comprising the amino acid sequence -Xaa-Xbb-Xcc-Xdd- or -Xbb-Xcc-Xdd-Xaa-, a protein hydrolysate which comprises said peptide, or a peptide mixture comprising said peptide is introduced into the nutraceutical, dietary supplement, food or feed product.

### DETAILED DESCRIPTION OF THE INVENTION

As shown in the present application the novel peptides which have a length of between 4 and 8 amino acids and comprising the amino acid sequence -Xaa-Xbb-Xcc-Xdd- or -Xbb-Xcc-Xdd-Xaa- are quite effective in lowering the cholesterol uptake in the human gastroinstestinal tract. It is found that the novel peptides, which have a length of between 4 and 8 amino acids and comprising the amino acid sequence -Xaa-Xbb-Xcc-Xdd- or -Xbb-Xcc-Xdd-Xaa-, resist gastrointestinal proteolytic degradation and are thus expected to be stable in the human intestinal tract. The novel peptides which have a length of between 4 and 8 amino acids and comprising the amino acid sequence -Xaa-Xbb-Xcc-Xdd- or -Xbb-Xcc-Xdd-Xaa- are therefore very suitable for an effective lowering cholesterol uptake and can for example be used in functional food, as a nutraceutical or as a medicament.

We noted that when the peptides of the invention were used in combination with plant sterols or green tea extract or epigallocatechin gallate, this cholesterol uptake lowering effect was even enhanced.

Although the present invention is not restricted to any theory or explanation, a possible explanation of the effect is that the peptide which is between 4 and 8 amino acids long and comprise the amino acid sequence -Xaa-Xbb-Xcc-Xdd- or -Xbb-Xcc-Xdd-Xaa- is capable of preventing cholesterol uptake in the gastrointestinal tract. The peptide which is between 4 and 8 amino acids long and comprise the amino acid sequence -Xaa-Xbb-Xcc-Xdd- or -Xbb-Xcc-Xdd-Xaa- is found to be stable in the gastroinstestinal tract which makes this peptide very suitable for having its cholesterol uptake decreasing effect all along the duodenum, the jejunum and the ileum. The peptide has preferably at least two positive charges due to the presence of least two amino acids selected from the group consisting of Arg (R) residue, Lys (K) residue or His (H) residue in its sequence. The remaining amino acids are preferably Pro (P) or Gly (G)

Examples of Foods for Special Nutritional Uses include the categories of sport foods, slimming foods, infant formula and clinical foods. The term dietary supplement as used herein denotes a product taken by mouth that contains a compound or mixture of compounds intended to supplement the diet. The compound or mixture of compounds in these products may include: vitamins, minerals, herbs or other botanicals and amino acids. Dietary supplements can also be extracts or concentrates, and may be found in many forms such as tablets, capsules, softgels, gelcaps, liquids, or powders. The term nutraceutical as used herein denotes the usefulness in both the nutritional and pharmaceutical field of application. The nutraceutical compositions according to the present invention may be in any form that is suitable for administrating to the animal body including the human body, especially in any form that is conventional for oral administration, e.g. in solid form such as (additives/supplements for) food or feed, food or feed premix, tablets, pills, granules, dragées, capsules, and effervescent formulations such as powders and tablets, or in liquid form such as solutions, emulsions or suspensions as e.g. beverages, pastes and oily suspensions. Controlled (delayed) release formulations incorporating the hydrolysates according to the invention also form part of the invention. Furthermore, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

Thus, novel nutraceutical compositions comprising the novel peptides which have a length of between 4 and 8 amino acids and comprising the amino acid sequence -Xaa-Xbb-Xcc-Xdd- or -Xbb-Xcc-Xdd-Xaa- can find use as supplement to food and beverages and as pharmaceutical formulations or medicaments for enteral or parenteral application which may be solid formulations such as capsules or tablets, or liquid formulations, such as solutions, suspensions or emulsions.

It is now found that the peptide according to the invention may be used as an ingredient in various food products. Such products may be pasteurised or even be sterilized to guarantee adequate shelf stabilities without dramatic losses of the relevant peptide activity. In food products such as condiments, various beverages and emulsions such as low fat spreads, the peptide remains sufficiently active to achieve an adequate cholesterol lowering effect. Therefore, the peptides according to the invention are advantageously combined with a large variety of food products, including those products incorporating cholesterol lowering stanols in their fat phase.

In general the taste of the food product is not affected or altered by the presence of the peptide With the necessary precautions, the peptide survives the sterilisation or pasteurization treatment and maintains its activity during the whole shelf life of the product.

The invention thus relates to sterilized, pasteurized and shelf stable food products containing the peptide of the invention.

A food product having an extended or improved shelf life is understood as having a shelf life of at least one week up to a year or more, during which the organoleptical properties as well as the microbial safety of the product are guaranteed. Obviously the allowable shelf life strongly depends on the actual storage conditions of the food product. Many perishable food products have to be stored cool in order to maximize their shelf lifes. Preferred food products are various types of dairy products. Dairy products are typically stored cool (preferably between 0 and 10 degrees Celsius) and comprise an aqueous as well as an fat phase hereby allowing the combination of the peptides according to the invention with hydrophobic stanols. Moreover, some of the peptides according to the invention can be obtained from various types of milk and easily combined with dairy derived calcium to create synergy. Other preferred food products are condiments containing the peptide according to the invention. Condiments are food products used to enhance the flavour of other foods. Condiments have the advantage of being abundantly present at home and in restaurants, diners and supermarkets, and typically have of a prolonged shelf life. Yet another category of preferred food products are different types of beverages. Examples of beverages which are perfectly suitable as carriers for the present peptides are fruit juices and fruit concentrates. In fact even acidified or carbonated bottled water would present an excellent carrier for the peptides. “Shots” like vegetable or fruit concentrates also fall within this category. Likewise acid products containing a food grade preservative like benzoate or sorbate present excellent carriers for the peptides. Also very acid products that require no pasteurisation at all, such as cola's, present excellent carriers.

Food products that are especially preferred as a carrier for the peptide of the invention are those food products that allow a combination of the peptide according to the invention with green tea extracts such as epigallocatechin gallate or with calcium salts such as calcium phosphate or with plant sterols or stanols or esterified stanols. Such preferred products include oil-in-water (O/W) or water-in-oil (W/O) emulsions. Typical examples of such emulsions are spreads, preferably a margarine or a low fat spread. The peptide of the invention can also be consumed in the form of a beverage, a powder, a pill or a tablet to give the desired cholesterol lowering effect.

The novel peptides which have a length of between 4 and 8 amino acids and comprising the amino acid sequence -Xaa-Xbb-Xcc-Xdd- or -Xbb-Xcc-Xdd-Xaa- can be produced by various methods including economically viable production routes. Relevant options to produce the peptides according to the invention are the hydrolysis of suitable proteins using selected enzymes as illustrated below for kappa casein of cow or dromedary milk. Preferably the peptide fraction is further purified from the hydrolysate by advantageously using the characteristics of the desired peptide. Known methods such as ultrafiltration, chromatography, precipitation or extraction procedures rely on molecular size, charge and solubilities. Yield of the desired peptide fractions can also be enhanced by integrating multimers of nucleotide sequences encoding the desired peptide into the genome of the organism producing the relevant substrate protein. An example of this approach was recently published for the hypocholesterolemic Ile-Ala-Glu-Lys peptide from beta-lactoglobulin (Prak et al., 2007, Peptides 27, 1179-1186). Another option is presented by the chemical synthesis of the desired peptides. Production via chemical synthesis is possible using conventional techniques as for instance described in “Peptides: Chemistry and Biology” by N. Sewald and H. D. Jakubke, Eds. Wiley-VCH Verlag GmbH, 2002, Chapter 4. Particular cost-effective methods of chemical peptide synthesis suitable for large-scale production are based on the use of alkylchloroformates or pivaloyl chloride for the activation of the carboxylic group combined with the use of methyl esters for C-terminal protection and benzyloxycarbonyl (Z) or tert-butyloxycarbonyl groups for N-protection. Furthermore chemo-enzymatic peptide synthesis presents an interesting option because of the favorable cost structure that can be obtained by this approach. Also peptide fermentation and non-ribosomal peptide synthesis present interesting options in this context.

Therefore, the novel peptides which have a length of between 4 and 8 amino acids and comprising the amino acid sequence -Xaa-Xbb-Xcc-Xdd- or -Xbb-Xcc-Xdd-Xaa- may be made by enzymatic hydrolysis or by fermentative approaches using any protein substrate containing the amino acid sequences -Xaa-Xbb-Xcc-Xdd- or -Xbb-Xcc-Xdd-Xaa-. Advantageously the protein substrate contains the fragments -Xaa-Xbb-Xcc-Xdd- or -Xbb-Xcc-Xdd-Xaa-. Collagens from cow, pig and chicken provide preferred protein substrates to generate K-G-K-P containing hydrolysates, the proline-rich protein from cotton is a preferred substrate to generate K-P-K-P containing hydrolysates, triticale is a preferred substrate to generate R-G-R-G containing hydrolysates, wheat phytase is a preferred substrate to generate P-R-P-K, collagen from cows is a preferred substrate to generate P-K-G-K, collagen from chicken is a preferred substrate to generate P-R-G-R, triticale is a preferred substrate to generate G-R-G-R, collagen from pigs is a preferred substrate to generate G-K-G-K, the proline-rich fraction of cotton is a preferred substrate to generate P-K-P-K, conglycinin from soy is a preferred substrate to generate P-R-P-R. Instead of collagen, preferably gelatin derived from the relevant source, for example cow, pig or chicken collagen, is used. The kappa casein fraction from cow milks or the milks of the dromedary or the guanaco represents another preferred protein substrates for such enzymatic or fermentative approaches. The sequence present in the kappa casein fraction of cow milk is: -R-H-P-H-P-H-L-S-F-M-A-I-P-P-K-K-

Hydrolysis with trypsin will yield H-P-H-P-H-L-S-F-M-A-I-P-P-K-, hydrolysis with trypsin plus chymosin will yield H-P-H-P-H-L-S-F. Evidently also peptide H-P-H-P is contained in the latter sequence.

The sequence present in the kappa casein fraction of dromedary milk is: -E-R-R-P-R-P-R-P-S-F-I-A-I-P-P-K-I Hydrolysis with a trypsine will result in R-P-R-P-R-P-S-F-I-A-I-P-P-K (14 amino acids, having a double carboxy terminal proline).

Hydrolysis with trypsine plus a subtilisin such as Alcalase or Protex will result in the formation of R-P-R-P-R-P-S-F (8 amino acids). Yet another approach is possible in which camel chymosin is used to cleave immediately C-terminal of the desired R-P-R-P-R-P-S sequence. Evidently also peptide R-P-R-P is contained in the latter sequence.

Because of the inherent resistancy of the peptides according to the invention against proteolytic attack, the desired peptides also can be obtained by approaches in which the less relevant parts of the protein substrate are removed. This can be accomplished by using complex proteolytic mixtures such as Sumizyme FP (Shin Nihon) or Flavourzyme (Novozymes) that will truncate larger peptides to yield the proteolytically resistant peptides according to the invention. Alternatively the relevant protein substrates can be subjected to a fermentation with various types of lactobacilli or streptococci. Through optimisation of the fermentation or hydrolysis conditions, the production of the biologically active peptides which have a length of between 4 and 8 amino acids and comprising the amino acid sequence -Xaa-Xbb-Xcc-Xdd- or -Xbb-Xcc-Xdd-Xaa- may be maximised. The skilled person trying to maximise the production will know how to adjust the process parameters, such as hydrolysis/fermentation time, hydrolysis/fermentation temperature, enzyme/microorganism type and concentration etc.

The peptides which have a length of between 4 and 8 amino acids and comprising the amino acid sequence -Xaa-Xbb-Xcc-Xdd- or -Xbb-Xcc-Xdd-Xaa- or compositions comprising these peptides are advantageously hydrolysates and preferably made according to a process involving the following steps:

(a) enzymatic hydrolysis of a suitable protein substrate comprising -Xaa-Xbb-Xcc-Xdd- or -Xbb-Xcc-Xdd-Xaa- in its amino acid sequence resulting in a hydrolysed protein product comprising the peptides which have a length of between 4 and 8 amino acids and comprising the amino acid sequence -Xaa-Xbb-Xcc-Xdd- or -Xbb-Xcc-Xdd-Xaa-;
(b) separation from the hydrolysed protein product of a fraction rich in peptides which have a length of between 4 and 8 amino acids and comprising the amino acid sequence -Xaa-Xbb-Xcc-Xdd- or -Xbb-Xcc-Xdd-Xaa-; and optionally
(c) concentrating and/or drying the hydrolysed protein product or a fraction rich in the desired peptides to obtain a concentrated liquid or a solid rich in peptides which have a length of between 4 and 8 amino acids and comprising the amino acid sequence -Xaa-Xbb-Xcc-Xdd- or -Xbb-Xcc-Xdd-Xaa-.

The enzymatic hydrolysis step may be any enzymatic treatment of the suitable protein substrate leading to hydrolysis of the protein resulting in liberation of peptides which have a length of between 4 and 8 amino acids and comprising the amino acid sequence -Xaa-Xbb-Xcc-Xdd- or -Xbb-Xcc-Xdd-Xaa-. As mentioned above several enzyme combinations can be used to release the desired peptides from the protein substrate. A suitable protein substrate may be any substrate encompassing the amino acid sequence -Xaa-Xbb-Xcc-Xdd- or -Xbb-Xcc-Xdd-Xaa-. In addition to the protein substates already mentioned, protein substrates known to encompass the peptides according to the invention can be obtained from, for example collagen, triticale, cotton, the latex of the para rubber tree, mouse ear cress, and from the garden pea.

The enzyme may be any enzyme or enzyme combination that is able to hydrolyse protein resulting in the liberation of one or more of the peptides of the invention.

The separation step (b) may be executed in any way known to the skilled person, e.g. by precipitation, filtration, centrifugation, extraction or chromatography and combinations thereof. Preferably the separation step (b) is executed using micro- or ultrafiltration techniques. The pore size of the membranes used in the filtration step, as well as the charge of the membrane may be used to control the separation of the peptides of the invention. Another preferred separation step is cation chromatography in which use is made of the positive charge of the peptide according to the invention under acid pH conditions. The concentration step (c) may involve nanofiltration or evaporation of the fraction generated by step (b) to yield a highly concentrated liquid.

If suitably formulated, e.g. with a low water activity (Aw), a low pH and preferably a preservative such as benzoate or sorbate, such concentrated liquid compositions form an attractive way of storage of the peptides according to the invention. Optionally the evaporation step is followed by a drying step e.g. by spray drying or freeze drying to yield a solid containing a high concentration of the peptides of the invention.

The enzymatic process comprises preferably a single enzyme incubation step. The process according to the invention has in general an incubation time of less than 24 hours, preferably the incubation time is less than 10 hours and more preferably less than 4 hours. The incubation temperature is in general higher than 30° C., preferably higher than 40° C. and more preferably higher than 50° C.

By protein hydrolysate, hydrolysate or hydrolysed protein is meant the product that is formed by enzymatic hydrolysis of the protein, an enriched hydrolysate being a fraction of the protein hydrolysate for example enriched in selected peptides or wherein peptides or polypeptides have been removed from the hydrolysate. So an enriched hydrolysate is preferably a mixture of peptides (or a peptide mixture). The peptide mixture of the invention is therefore a mixture of at least two peptides whereof at least one is a peptide comprising the amino acid sequence -Xaa-Xbb-Xcc-Xdd- or -Xbb-Xcc-Xdd-Xaa.

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” is defined herein as a chain containing more than 30 amino acid residues. All (oligo)peptide and polypeptide formulas or sequences herein are written from left to right in the direction from amino-terminus to carboxy-terminus, in accordance with common practice. The one-letter code of amino acids used herein is commonly known in the art and can be found in Sambrook, et al. (Molecular Cloning: A Laboratory Manual, 2nd, ed. Cold Spring Harbor Laboratory, Cold Spring Harbor Laboratory Press, Cold Spring Harbor, N.Y., 1989). An endoprotease is defined herein as an enzyme that hydrolyses peptide bonds in a polypeptide in an endo-fasion and belongs to the group EC 3.4. The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25). Exoproteases are defined herein as enzymes that hydrolyze peptide bonds adjacent to a terminal α-amino group (“aminopeptidases”), or a peptide bond between the terminal carboxyl group and the penultimate amino acid (“carboxypeptidases”). To prepare the hydrolysates incorporating the peptides according to the present invention the endoproteases trypsin (EC 3.4.21.4), subtilisin (EC 3.4.21.62) and proline-specific endoproteases such as prolyl oligopeptidase (EC 3.4.21.26) and the proline-specific endoprotease from A. niger (WO 02/45524) are preferred. Truncation of the peptides thus produced can be achieved with a suitable amino- and/or carboxypeptidase.

According to the present process preferably at least 20%, more preferably at least 30%, most preferably at least 40% of an -Xaa-Xbb-Xcc-Xdd- or -Xbb-Xcc-Xdd-Xaa- containing protein sequence is converted into peptides which have a length of between 4 and 8 amino acids and comprising the amino acid sequence -Xaa-Xbb-Xcc-Xdd- or -Xbb-Xcc-Xdd-Xaa-.

After elution from the resin, decantation, filtration or low speed centrifugation, the liquids containing the biologically active peptides, an enriched hydrolysate, can be recovered in a purified state. A subsequent evaporation and spray drying step will yield an economical route for obtaining a food grade concentrate, paste or powder with a high bio-activity. The proper formulation of such a concentrate for example by lowering the water activity (Aw) in combination with a pH adjustment or the addition of a food grade preservative like a benzoate or a sorbate will yield a microbiologically stabilized, food grade, liquid concentrate of the peptides of the invention. If required, the supernatant obtained after the decantation, filtration or low speed centrifugation can be further processed to improve the palatability of the final product. For example, the supernatant can be contacted with powdered activated charcoal followed by a filtration step to remove the charcoal. If required, the concentration of the bioactive ingredients can be increased even further by subsequent purification steps in which use is made of the specific amino acid composition of the peptides of the invention. We have found that peptides which have a length of between 4 and 8 amino acids and comprising the amino acid sequence -Xaa-Xbb-Xcc-Xdd- or -Xbb-Xcc-Xdd-Xaa- survive incubation conditions simulating the digestive conditions typically found in the gastro-intestinal tract. On the basis of these data we conclude that the novel peptides are likely to survive in the mammalian (for example human) gastrointestinal tract implying a considerable economic potential if used to treat the health risks typically associated with increased plasma cholesterol levels.

The peptides which have a length of between 4 and 8 amino acids and comprising the amino acid sequence -Xaa-Xbb-Xcc-Xdd- or -Xbb-Xcc-Xdd-Xaa- as obtained either before or after an additional (for example chromatographic) purification step may be used for the incorporation into food products that are widely consumed on a regular basis. Examples of such products are margarines, spreads, various dairy products such as butter or yoghurts or milk or whey containing beverages. Although such compositions are typically administered to human beings, they may also be administered to animals, preferably mammals, to counteract high cholesterol levels. Furthermore the high concentration of peptides suitable for lowering cholesterol uptake, makes these products very useful for the incorporation into dietary supplements in the form off pills, tablets or highly concentrated solutions or pastes or powders. Slow release dietary supplements that will ensure a continuous release of these peptides are of particular interest. The peptides which have a length of between 4 and 8 amino acids and comprising the amino acid sequence -Xaa-Xbb-Xcc-Xdd- or -Xbb-Xcc-Xdd-Xaa- according to the invention may be formulated as a dry powder in, for example, a pill, a tablet, a granule, a sachet or a capsule. Alternatively the peptides according to the invention may be formulated as a liquid in, for example, a syrup or a capsule. The compositions used in the various formulations and containing the peptides according to the invention may also incorporate at least one compound of the group consisting of a physiologically acceptable carrier, adjuvant, excipient, stabiliser, buffer and diluant which terms are used in their ordinary sense to indicate substances that assist in the packaging, delivery, absorption, stabilisation, or, in the case of an adjuvant, enhancing the physiological effect of the peptides, such as catechins or calcium salts. The relevant background on the various compounds that can be used in combination with the peptides according to the invention in a powdered form can be found in “Pharmaceutical Dosage Forms”, second edition, Volumes 1, 2 and 3, ISBN 0-8247-8044-2 Marcel Dekker, Inc.

Preferably, the nutraceutical or food product comprises from 2 to 30 wt % plant-derived sterol, more preferably from 4 to 20 wt % plant-derived sterol. Incorporation of plant-derived sterol maximizes reductions in LDL-cholesterol achieveable by dietary means. Sterols represent a group of compounds that are alcoholic derivatives of cyclopentanoperhydrophenanthrene and are an essential constituent of cell membranes in animals and plants (A. H. Lichtenstein, Circulation, 2000, 103, p. 1117). Cholesterol is the sterol of mammalian cells, whereas multiple sterols, or phytosterols, are produced by plants, with sitosterol, campesterol, and stigmasterol being most common. Plant sterols, although structurally similar to cholesterol, are not synthesized by the human body. They are very poorly absorbed by the human intestine. The specific plant sterols that are currently incorporated into foods intended to lower blood cholesterol levels are extracted from soybean oil or tall (pine tree) oil. Additional sources of plant sterols may be available in the near future. The plant sterols currently incorporated into foods are esterified to unsaturated fatty acids (creating sterol esters) to increase lipid solubility, thus allowing maximal incorporation into a limited amount of fat. Some plant sterols currently available are saturated, to form the stanol derivatives, sitostanol and campestanol, which after esterification form stanol esters. These stanols interfere with small intestine absorption of intestinal and biliary cholesterol. While they lower LDL (Low Density Lipoprotein) levels, they have no significant effect on HDL (High Density Lipoprotein) or triglyceride levels (N Eng J Med 1995; 333: 1308-1312). Quite surprisingly our data (see Examples 8 and 9) demonstrate that the peptides according to the invention have the capacity to significantly increase HDL plasma levels. Diabetes patients as well as physically inactive individuals persons and smokers are known to suffer from low HDL levels. Using the peptides according to the present invention compensates for such low HDL levels. Also some drugs like beta blockers, thiazide, diuretica and anabol steroids may result in lowered plasma HDL level. For all these persons the present invention offers a solution to increase the plasma HDL level. We herewith want to make a note that of course we do not want encourage unhealthy life styles like smoking or remaining physically inactive.

The above described nutraceutical ingredients contributing to increasing cardiovascular health as well as K+, Ca2+ and Mg2+, B-vitamins (folic acid, B6, B12) and sterols/stanols are herein collectively referred to as heart health ingredients.

### Materials and Methods

### Materials

All synthetic peptides used in the following Examples were obtained from Pepscan Presto B.V., Lelystad, The Netherlands.

### Methods

LC/MS/MS analysis used in the detection of peptides with a length of between 4 and 8 amino acids and comprises the amino acid sequence -Xaa-Xbb-Xcc-Xdd- or -Xbb-Xcc-Xdd-Xaa-.

HPLC using an ion trap mass spectrometer (Thermoquest®, Breda, the Netherlands) coupled to a P4000 pump (Thermoquest®, Breda, the Netherlands) was used in quantification of the peptides of interest in the enzymatic protein hydrolysates produced. The peptides formed were separated using a Inertsil 3 ODS 3, 3 μm, 150*2.1 mm (Varian Belgium, Belgium) column in combination with a gradient of 0.1% formic acid in Milli Q water (Millipore, Bedford, Mass., USA; Solution A) and 0.1% formic acid in acetonitrile (Solution B) for elution. The gradient started at 100% of Solution A, kept here for 5 minutes, increasing linear to 5% B in 10 minutes, followed by linear increasing to 45% of solution B in 30 minutes and immediately going to the beginning conditions, and kept here 15 minutes for stabilization. The injection volume used was 50 microliters, the flow rate was 200 microliter per minute and the column temperature was maintained at 55° C. The protein concentration of the injected sample was approx. 50 micrograms/milliliter.

Detailed information on the individual peptides was obtained by using dedicated MS/MS for the peptides of interest, using optimal collision energy of about 30%. Quantification of the individual peptides was performed using external calibration, by using the most abundant fragment ions observed in MS/MS mode.

Prior to LC/MS/MS the enzymatic protein hydrolysates were centrifuged at ambient temperature and 13000 rpm for 10 minutes, filtered through a 0.22 μm filter and the supernatant was diluted 1:100 with MilliQ water.

Kjeldahl Nitrogen

Total Kjeldahl Nitrogen was measured by Flow Injection Analysis. Using a Tecator FIASTAR 5000 Flow Injection System equipped with a TKN Method Cassette 5000-040, a Pentium 4 computer with SOFIA software and a Tecator 5027 Autosampler the ammonia released from protein containing solutions was quantitated at 590 nm. A sample amount corresponding with the dynamic range of the method (0.5-20 mg N/l) is placed in the digestion tube together with 95-97% sulphuric acid and a Kjeltab subjected to a digestion program of 30 minutes at 200 degrees C. followed by 90 minutes at 360 degrees C. After injection in the FIASTAR 5000 system the nitrogen peak is measured from which the amount of protein measured can be inferred.

Amino Acid Analysis

A precisely weighed sample of the proteinaceous material was dissolved in dilute acid and precipitates were removed by centrifugation in an Eppendorf centrifuge. Amino acid analysis was carried out on the clear supernatant according to the PicoTag method as specified in the operators manual of the Amino Acid Analysis System of Waters (Milford Mass., USA). To that end a suitable sample was obtained from the liquid, then dried and subjected to vapour phase acid hydrolysis and derivatised using phenylisothiocyanate. The various derivatised amino acids present were quantitated using HPLC methods and added up to calculate the total level of free amino acids in the weighed sample. The amino acids Cys and Trp are not included in the data obtained in this analysis.

The Degree of Hydrolysis (DH) of the protein hydrolysate was measured using a rapid OPA test and calculated as described (Nielsen et al, JFS, Vol 66, NO 5, 642-646, 2001).

Molecular weight distribution of peptides and proteins present in hydrolysates. Analysis of the peptide size distribution of protease treated protein samples was done on an automated HPLC system equipped with a high pressure pump, injection device able to inject 10-100 μl sample and a UV detector able to monitor the column effluent at 214 nm. The column used for this analysis was a Superdex Peptide HR 10/300 GL (Amersham) equilibrated with 20 mM Sodium Phosphate/250 mM Sodium Chloride pH 7.0 buffer. After injecting a sample (typically 50 μl) the various components were eluted from the column with buffer in 90 min at a flow rate of 0.5 ml/min. The system was calibrated using a mixture of cytochrome C (Mw 13 500 Da), aprotinin (Mw 6510 Da) and tetra-glycine (Mw 246 Da) as molecular weight markers.

The following Examples illustrate the invention further.

### EXAMPLES

### Example 1

### Method for Obtaining a Peptide According to the Invention from Camel Milk

To camel milk recombinant camel chymosin is added (Kappeler et al., BBRC 342 (2006) 647-654) to precipitate the caseinates and to cleave the soluble kappa-casein in a soluble glycomacroprotein part and an acid insoluble para-kappa casein part. The para-kappa casein part incorporates peptide RPRPRPS according to the invention. After centrifugation the caseinates incorporating the para-kappa casein is obtained. Peptide RPRPRPSF can be obtained upon redissolving the pellet by adding NaOH to a pH around 8 followed by a prolonged incubation with the endoproteases trypsin (Novozymes, Denmark) and subtilisin (Alcalase from Novozymes, Denmark). After a thermal inactivation of the two enzymes used, peptide RPRPRPSF can be partially purified from the resulting hydrolysate by cation chromatography in which the positive charge of this arginine-rich peptide is used as the selection criterion.

### Example 2

### Testing the In Vivo Efficacy of Peptide RPRPRPSF with and without Beta-Sitosterol

Peptide RPRPRPSF is chemically synthesized and beta-sitosterol is obtained from Sigma. Both compounds, apart and in combination, are subjected to a testing protocol which is essentially similar to the in vivo testing protocol described by Nagaoka et al., BBRC 281 (2001) 11-17. Similar to the results obtained by Nagaoka et al., peptide RPRPRPSF shows a hypercholesterolemic effect that can be further enhanced by combination with beta-sitosterol.

### Example 3

### Soft Gelatin Capsule

Soft gelatin capsules are prepared by conventional procedures using ingredients specified below:

Active ingredients: a peptide which is between 4 and 8 amino acids long and comprise the amino acid sequence -Xaa-Xbb-Xcc-Xdd- or -Xbb-Xcc-Xdd-Xaa-0.1 g, protein hydrolysates 0.3 g
Other ingredients: glycerol, water, gelatin, vegetable oil.

### Example 4

### Hard Gelatin Capsule

Hard gelatin capsules are prepared by conventional procedures using ingredients specified below:

Active ingredients: a peptide which is between 4 and 8 amino acids long and comprise the amino acid sequence -Xaa-Xbb-Xcc-Xdd- or -Xbb-Xcc-Xdd-Xaa-0.3 g, protein hydrolysates 0.7 g
Fillers: lactose or cellulose or cellulose derivatives q.s
Lubricant: magnesium stearate if necessary (0.5%)

### Example 5

### Tablet

Tablets are prepared by conventional procedures using ingredients specified below:

Active ingredients: a peptide which is between 4 and 8 amino acids long and comprise the amino acid sequence -Xaa-Xbb-Xcc-Xdd- or -Xbb-Xcc-Xdd-Xaa-0.4 g, unhydrolysed protein 0.4 g
Other ingredients: microcrystalline cellulose, silicone dioxide (SiO₂), magnesium stearate, crosscarmellose sodium.

### Example 6

### Soft Drink with 30% Juice

Typical serving: 240 ml

### Active Ingredients:

a peptide which is between 4 and 8 amino acids long and comprise the amino acid sequence -Xaa-Xbb-Xcc-Xdd- or -Xbb-Xcc-Xdd-Xaa- and protein hydrolysates and maltodextrin as a carbohydrate source are incorporated in this food item:
A peptide which is between 4 and 8 amino acids long and comprise the amino acid sequence -Xaa-Xbb-Xcc-Xdd- or -Xbb-Xcc-Xdd-Xaa-: 0.5-5 g/per serving
Protein hydrolysates: 1.5-15 g/per serving
Maltodextrin: 3-30 g/per serving
1.0 A Soft Drink Compound is Prepared from the Following Ingredients

### Juice Concentrates and Water Soluble Flavors:

| | [g] |
|---|---|
| 1.1 Orange concentrate | |
| 60.3 Brix, 5.15% acidity | 657.99 |
| Lemon concentrate | |
| 43.5 Brix, 32.7% acidity | 95.96 |
| Orange flavor, water soluble | 13.43 |
| Apricot flavor, water soluble | 6.71 |
| Water | 26.46 |
| 1.2 Color | |
| β-Carotene 10% CWS | 0.89 |
| Water | 67.65 |
| 1.3 Acid and Antioxidant | |
| Ascorbic acid | 4.11 |
| Citric acid anhydrous | 0.69 |
| Water | 43.18 |
| 1.4 Stabilizers | |
| Pectin | 0.20 |
| Sodium benzoate | 2.74 |
| Water | 65.60 |
| 1.5 Oil soluble flavors | |
| Orange flavor, oil soluble | 0.34 |
| Orange oil distilled | 0.34 |

1.6 Active Ingredients

Active ingredients (this means the active ingredient mentioned above: a peptide which is between 4 and 8 amino acids long and comprise the amino acid sequence -Xaa-Xbb-Xcc-Xdd- or -Xbb-Xcc-Xdd-Xaa-) and protein hydrolysates and maltodextrin in the concentrations mentioned above.

Fruit juice concentrates and water soluble flavors are mixed without incorporation of air. The color is dissolved in deionized water. Ascorbic acid and citric acid is dissolved in water. Sodium benzoate is dissolved in water. The pectin is added under stirring and dissolved while boiling. The solution is cooled down. Orange oil and oil soluble flavors are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenizing using a Turrax and then a high-pressure homogenizer (p₁=200 bar, p₂=50 bar).

2.0 A Bottling Syrup is Prepared from the Following Ingredients:

| | [g] |
|---|---|
| Softdrink compound | 74.50 |
| Water | 50.00 |
| Sugar syrup 60 Brix | 150.00 |

The ingredients of the bottling syrup are mixed together. The bottling syrup is diluted with water to 1 l of ready to drink beverage.

### Variations:

Instead of using sodium benzoate, the beverage may be pasteurized. The beverage may also be carbonized.

### Example 7

### Effects of Peptides on the Solubility of Cholesterol in Mixed Micelles

The mixed micelles were prepared according to the method described by Nagaoka et al. (Biochemical and Biophysical Research Communications 281: 11-17 (2001)) by sonication of a 15 mM phosphate buffer saline (PBS) solution containing 6.6 mM taurocholate, 2.4 mM lecithin, 0.5 mM cholesterol and either 10 mg/ml of octapeptide (RPRPRPSF, MW1012) or 10 mg/ml soy protein (SUPRO 760, Solea Belgium) at pH 7.4. The lipids, phospholipids and cholesterol were dissolved in methanol and dried before adding the PBS buffer and the peptides or the soy protein at a final concentration of 10 mg/ml. The resulting micellar solution was kept at 37° C. for 24 hours and centrifuged at 1000×g for 10 min. In order to separate any precipitated cholesterol from the intramicellar cholesterol the solution was then filtered through a 0.22 μm MILLEX-GP filter (Millipore, Bedford, Mass., USA). The intramicellar concentration of cholesterol was then determined enzymatically using a cholesterol kit and a Hitachi 912 automatic analyzer from Roche Diagnostica, Switzerland.

TABLE 1
Cholesterol content of the micellar solution

| | Cholesterol (mmol/L) |
|---|---|
| Control | 0.494 |
| Octapeptide (10 mg/ml) | 0.153 |
| Supro 760 (10 mg/ml) | 0.427 |

According to the data presented in Table 1, the intramicellar concentration of cholesterol drastically decreased in the presence of 10 mg/ml of the octapeptide (RPRPRPSF). In vivo the principal steps in the absorption of cholesterol are emulsification in the stomach, hydrolysis of the ester bond by a specific pancreatic esterase, micellar solubilization, absorption in the proximal jejunum, re-esterification within the intestinal cells, and transport to the lymph by chylomicrons. Due to the insolubility of cholesterol in water, solubilization of cholesterol in mixed micelles is a requirement for its efficient absorption. Thus, the observed effect of octapeptide (RPRPRPSF) in minimizing the incorporation of cholesterol in micelles, could reduce cholesterol absorption from the intestine which may lead to decreased blood cholesterol levels.

### Example 8

### Hypocholesterolemic Effects of Peptides in Fisher F344 Rats

Forty male Fisher rats (F344) from Charles River (Sulzfeld, Germany), weighing 70-85 g were housed individually with free access to water and feed, with an alternating 12-hour light-dark cycle. The animals were fed a rodent atherogenic diet high in cholesterol and fat for the 2 weeks preceding the treatment. The macro-nutrient composition of the diet was as follows (g/100 g anhydrous mix): protein 20.8; fiber 4.2; fat 15; carbohydrate 48. The diet also contained 1.25 wt % cholesterol, 0.5 wt % sodium cholate and a standard vitamin and mineral mix, prepared according to rat nutritional requirements. The main fats consisted of cacoa butter (7.5 wt %) and soja oil (4.35 wt %). After 2 weeks of adaptation to the diet the rats were randomized according to body weight into four experimental groups of 10 animals. The four groups were treated orally every morning for 7 days with either water (control group) or octapeptide (RPRPRPSF) at two doses of 200 and 800 mg/kg body weight (BW) respectively or soy protein (SUPRO 760) at 800 mg/kg BW while continuing the aetherogenic diet. There was no difference in feed consumption over the whole experiment. The day before the last administration, all animals were dosed orally in the evening and deprived of food overnight (14 hours) but allowed free access to water. At day 7 the fasted rats were treated again orally in the morning and after two hours the animals were sacrificed by withdrawing blood from the vena Cava under Isoflurane anesthesia. Blood was collected into tubes containing EDTA as an anticoagulant. Plasma was prepared from the blood by immediate centrifugation at 1600 g for 10 minutes at 4° C. Assays of plasma cholesterol and HDL-cholesterol (precipitation method) were determined enzymatically on a Hitachi 912 automatic analyzer (Roche Diagnostica, Switzerland). Non-HDL cholesterol was calculated by difference between total cholesterol and HDL cholesterol. All data are expressed as means±standard deviation (SD) for animals in each diet group, with n being the number of rats. Statistical significance of the mean differences between dietary groups was tested by one-way analysis of variance (ANOVA). If significant differences were found, the Dunnett's test for multiple comparison was used to compare each group to the control group. P values less than 0.05 were considered significant (Table 2).

The octapeptide (RPRPRPSF) at 200 and 800 mg/Kg BW significantly lowered plasma total cholesterol levels and non-HDL cholesterol (VLDL-cholesterol+LDL-cholesterol) levels as compared to the control group. Plasma HDL-cholesterol was significantly increased after treatment with the octapeptide (RPRPRPSF) when compared to the control group. Thus the ratio of non-HDL cholesterol to HDL-cholesterol, a strong indicator of cardiovascular and atherosclerosis risk, was positively affected in the animals who were treated with the octapeptide (RPRPRPSF).

TABLE 2
Effects of peptides on plasma total cholesterol and lipoproteins cholesterol levels in rats^{a}

| | Total cholesterol | non HDL cholesterol | HDL cholesterol |
|---|---|---|---|
| Control | 12.8 ± 3.1 | 12.5 ± 3.1 | 0.22 ± 0.12 |
| Octapeptide (RPRPRPSF) | 4.1 ± 1.2^{b} | 3.6 ± 1.2^{b} | 0.54 ± 0.20^{b} |
| 200 mg/Kg | | | |
| Octapeptide (RPRPRPSF) | 3.4 ± 0.7^{b} | 2.9 ± 0.7^{b} | 0.51 ± 0.17^{b} |
| 800 mg/Kg | | | |
| Soy protein (SUPRO 760) | 11.6 ± 3.4 | 11.4 ± 3.4 | 0.24 ± 0.12 |
| 800 mg/Kg | | | |

^{a}Values are expressed as mean ± SD given in mmol/L (n = 10 per group)
^{b}significantly different from control (p < 0.05)

### Example 9

### Hypocholesterolemic Effects of Five Small Peptides in Fisher F344 Rats

In this experiment the effects of various penta-, and tetrapeptides were tested on plasma cholesterol levels in the hypercholesterolemic rat model.

In short eighty male Fisher rats (F344) from Charles River (Sulzfeld, Germany), weighing 65-96 g were housed individually with free access to water and feed, with an alternating 12-hour light-dark cycle. The animals were fed a rodent atherogenic diet high in cholesterol and fat for the 2 weeks preceding the treatment. The macro-nutrient composition of the diet was as follows (g/100 g anhydrous mix): protein 20.8; fiber 4.2; fat 15; carbohydrate 48. The diet also contained 1.25 wt % cholesterol, 0.5 wt % sodium cholate and a standard vitamin and mineral mix, prepared according to rat nutritional requirements. The main fats consisted of cacoa butter (7.5 wt %) and soja oil (4.35 wt %). After 2 weeks of adaptation to the diet the rats were randomized according to body weight into four experimental groups of 10 animals. The eight groups were treated orally every morning for 7 days with either water (control group) or one of the following peptides: RPRPS, RGRPS, HPHPH, RPRP, KPKPS, at a dose of 100 mg/kg body weight (BW) while continuing the aetherogenic diet. There was no difference in feed consumption over the whole experiment. The day before the last administration, all animals were dosed orally in the evening and deprived of food overnight (14 hours) but allowed free access to water. At day 7 the fasted rats were treated again orally in the morning and after two hours the animals were sacrificed by withdrawing blood from the vena Cava under Isoflurane anesthesia. Blood was collected into tubes containing EDTA as an anticoagulant. Plasma was prepared from the blood by immediate centrifugation at 1600 g for 10 minutes at 4° C. Assays of plasma cholesterol and HDL-cholesterol (precipitation method) were determined enzymatically on a Hitachi 912 automatic analyzer (Roche Diagnostica, Switzerland). Non-HDL cholesterol was calculated by difference between total cholesterol and HDL cholesterol. One animal in the control group was excluded from the analysis due to hemolytic plasma which interfered with the measurements. All data are expressed as means±standard deviation (SD) for animals in each diet group, with n being the number of rats. Statistical significance of the mean differences between dietary groups was tested by one-way analysis of variance (ANOVA). If significant differences were found, the Dunnett's test for multiple comparison was used to compare each group to the control group. P values less than 0.05 were considered significant (Table 3).

The five tested peptides (100 mg/Kg BW) significantly lowered plasma total cholesterol levels and non-HDL cholesterol (VLDL-cholesterol+LDL-cholesterol) levels as compared to the control group. Plasma HDL-cholesterol was significantly increased after treatment with the experimental peptides when compared to the control group. The data suggest that the peptides strongly reduce risk factors for cardiovascular problems and atherosclerosis. In a dose response experiment the effective dose for each of the five tested peptides can be determined.

TABLE 3
Effects of peptides on plasma total cholesterol
and lipoproteins cholesterol levels in rats^{a}

| | Total cholesterol | non HDL cholesterol | HDL cholesterol |
|---|---|---|---|
| Control | 10.1 ± 2.2 | 10.0 ± 2.2 | 0.14 ± 0.08 |
| RPRPS | 3.6 ± 1.1^{b} | 3.3 ± 1.2^{b} | 0.29 ± 0.06^{b} |
| RGRPS | 3.2 ± 0.4^{b} | 2.9 ± 0.4^{b} | 0.37 ± 0.14^{b} |
| HPHPH | 3.4 ± 1.2^{b} | 3.1 ± 1.3^{b} | 0.29 ± 0.13^{b} |
| RPRP | 2.9 ± 0.4^{b} | 2.6 ± 0.4^{b} | 0.33 ± 0.11^{b} |
| KPKPS | 3.0 ± 1.0^{b} | 2.7 ± 1.0^{b} | 0.29 ± 0.11^{b} |

^{a}Values are expressed as mean ± SD given in mmol/L (n = 9-10 per group)
^{b}significantly different from control (p < 0.05)

1. A peptide which has a length of between 4 and 8 amino acids, preferably between 4 and 6 amino acids and more preferably between 4 or 5 amino acids, and comprises the amino acid sequence -Xaa-Xbb-Xcc-Xdd- or -Xbb-Xcc-Xdd-Xaa-, whereby Xaa and Xcc can be His (H), Arg (R) or Lys (K), and Xbb and Xdd can be Pro (P) or Gly (G).

2. The peptide according to claim 1 which has a positive charge.

3. The peptide according to claim 1 whereby Xbb and/or Xdd is Pro (P).

4. The peptide according to claim 1 whereby Xaa and/or Xcc is Arg (R).

5. The peptide according to claim 1 which is -R-P-R-P-R-P-S-F-I-A-I-P-P-K- (SEQ ID NO:2), R-P-R-P-R-P-S-F (SEQ ID NO:3), R-P-R-P-R-P-S (SEQ ID NO:4), R-P-R-P-S (SEQ ID NO:5), R-P-R-P (SEQ ID NO:6), K-P-R-P (SEQ ID NO:9), R-G-R-P-S (SEQ ID NO:7), H-P-H-P-H (SEQ ID NO:8), H-P-H-P-H-L-S-F, K-P-K-P-S (SEQ ID NO:11), K-G-K-P (SEQ ID NO:12), K-P-K-P (SEQ ID NO:13), R-P-R-P (SEQ ID NO:14), R-G-R-G (SEQ ID NO:15), P-R-P-K (SEQ ID NO:16), P-K-G-K (SEQ ID NO:17), P-R-G-R- (SEQ ID NO:18), G-R-G-R (SEQ ID NO:19), G-K-G-K (SEQ ID NO:20), P-K-P-K (SEQ ID NO:21) and P-R-P-R (SEQ ID NO:22).

6. The peptide according to claim 1 which is a pharmaceutical active compound.

7. A protein hydrolysate comprising a peptide according to claim 1.

8. A protein hydrolysate according to claim 7 which has a DH of 5 to 50%, preferably 10 to 40%, more preferably a DH of 20 to 35%.

9. A peptide mixture comprising at least one peptide according to claim 1.

10. A peptide mixture of claim 9 which comprises at least 1 mg of said peptide/gram protein, preferably at least 4 mg/g, even more preferably at least 10 mg/g protein, still more preferably at least 25 mg/g protein and most preferably at least 50 mg/g protein.

11. A peptide mixture according to claim 9 whereby the amount of peptides which have a MW of less than 2000 Da is at least 30 wt % (dry matter) of the peptide mixture, preferably between 35 and 90% wt (dry matter) of the peptide mixture.

12. A peptide mixture according to claim 8.

13. A method to produce a peptide which has a length of between 4 and 8 amino acids, preferably between 4 and 6 amino acids and more preferably between 4 or 5 amino acids, and comprises the amino acid sequence -Xaa-Xbb-Xcc-Xdd- or -Xbb-Xcc-Xdd-Xaa-, whereby Xaa and Xcc can be His (H), Arg (R) or Lys (K), and Xbb and Xdd can be Pro (P) or Gly (G), which comprises the enzymatic hydrolysis of a suitable protein.

14. A method according to claim 12 which comprises the use of a protease to hydrolyze a suitable protein, preferably at least two endoproteases are used.

15. A method of treatment or preventing of hypercholesterolemia, artherosclerosis, coronary heart disease or increasing plasma HDL (High Density Lipoprotein) which comprises the intake of a peptide according to claim 1, a protein hydrolysate comprising said peptide, or a peptide mixture comprising at least said peptide or the intake of a nutraceutical, food or fee product which comprises said peptide, a protein hydrolysate comprising said peptide, or a peptide mixture comprising at least said peptide.

16. A method for the preparation of a nutraceutical, dietary supplement, food or feed product for the treatment or preventing of hypercholesterolemia, artherosclerosis, coronary heart disease or increasing plasma HDL (High Density Lipoprotein) whereby a peptide according to claim 1, a protein hydrolysate comprising said peptide, or a peptide mixture comprising at least said peptide is introduced in said nutraceutical, food or fee product.

17. A nutraceutical, dietary supplement, food or feed product for the treatment or preventing of hypercholesterolemia, artherosclerosis, coronary heart disease or increasing plasma HDL (High Density Lipoprotein) comprising a peptide according to claim 1, a protein hydrolysate comprising said peptide, or a peptide mixture comprising at least said peptide.


SEQUENCE LISTING (22 sequences)

SEQ ID NO: 1 - protein, 17 aa, Artificial Sequence
  synthetic peptide
ERRPRPRPSFIAIPPKI

SEQ ID NO: 2 - protein, 14 aa, Artificial Sequence
  synthetic peptide
RPRPRPSFIAIPPK

SEQ ID NO: 3 - protein, 8 aa, Artificial Sequence
  synthetic peptide
RPRPRPSF

SEQ ID NO: 4 - protein, 7 aa, Artificial Sequence
  synthetic peptide
RPRPRPS

SEQ ID NO: 5 - protein, 5 aa, Artificial Sequence
  synthetic peptide
RPRPS

SEQ ID NO: 6 - protein, 4 aa, Artificial Sequence
  synthetic peptide
RPRP

SEQ ID NO: 7 - protein, 5 aa, Artificial Sequence
  synthetic peptide
RGRPS

SEQ ID NO: 8 - protein, 5 aa, Artificial Sequence
  synthetic peptide
HPHPH

SEQ ID NO: 9 - protein, 5 aa, Artificial Sequence
  synthetic peptide
KPKPS

SEQ ID NO: 10 - protein, 4 aa, Artificial Sequence
  synthetic peptide
KPRP

SEQ ID NO: 11 - protein, 5 aa, Artificial Sequence
  synthetic peptide
KPKPS

SEQ ID NO: 12 - protein, 4 aa, Artificial Sequence
  synthetic peptide
KGKP

SEQ ID NO: 13 - protein, 4 aa, Artificial Sequence
  synthetic peptide
KPKP

SEQ ID NO: 14 - protein, 4 aa, Artificial Sequence
  synthetic peptide
RPRP

SEQ ID NO: 15 - protein, 4 aa, Artificial Sequence
  synthetic peptide
RGRG

SEQ ID NO: 16 - protein, 4 aa, Artificial Sequence
  synthetic peptide
PRPK

SEQ ID NO: 17 - protein, 4 aa, Artificial Sequence
  synthetic peptide
PKGK

SEQ ID NO: 18 - protein, 4 aa, Artificial Sequence
  synthetic peptide
PRGR

SEQ ID NO: 19 - protein, 4 aa, Artificial Sequence
  synthetic peptide
GRGR

SEQ ID NO: 20 - protein, 4 aa, Artificial Sequence
  synthetic peptide
GKGK

SEQ ID NO: 21 - protein, 4 aa, Artificial Sequence
  synthetic peptide
PKPK

SEQ ID NO: 22 - protein, 4 aa, Artificial Sequence
  synthetic peptide
PRPR